(19) United States
(12) Patent Application Publication
(10) Pub. No.: US 2019/0290560 A1
(43) Pub. Date: Sep. 26, 2019

(54) High SPF, Photo-protective Zinc Oxide Sunscreen Emulsions

(71) Applicant: Amavara, Inc., San Diego, CA (US)

(72) Inventor: Laura C. Singleton, Los Angeles, CA (US)

(21) Appl. No.: 16/220,370

(22) Filed: Dec. 14, 2018

Related U.S. Application Data
(60) Provisional application No. 62/598,848, filed on Dec. 14, 2017.

(51) Int. Cl. A61K 8/27; A61K 8/26; A61K 8/06; A61K 8/58; A61K 8/891; A61K 8/25; A61Q 17/04

(52) U.S. Cl. CPC A61K 8/27; A61K 8/26; A61K 8/064; A61K 8/585; A61K 2800/43; A61K 8/25; A61Q 17/04; A61K 2800/30; A61K 2800/262; A61K 8/891

(57) ABSTRACT

A broad spectrum, emulsion sunscreen composition having an SPF value of at least 30, wherein the composition (xx) does not contain an organic sunscreen active that absorbs ultraviolet radiation and (yy) contains only a physical sunscreen active ingredient, Zinc Oxide, having (i) a porous structure, such that when pores within Zinc Oxide particles are filled with an excipient, the refractive index of the Zinc Oxide particles is reduced; (ii) an average particle size of greater than about 100 nanometers, such that the Zinc Oxide is visibly transparent when the sunscreen composition is applied to skin; and (iii) is water resistant for 80 minutes as determined in accordance with the test method codified at 21 CFR § 201.327(i)(7).

### FIELD OF INVENTION

Disclosed are sunscreen emulsions (i) having a “Sun Protection Factor” (SPF) value of at least 30, preferably at least 50 (see definition below), that are (ii) “broad spectrum” (according to regulations promulgated by both the US Food and Drug Administration (FDA) and the European Commission; see discussion below), (iii) do not contain an organic sunscreen active that absorbs ultraviolet radiation (UVR) and (iv) contains only a physical sunscreen active ingredient—“non-nano” Zinc Oxide. For purposes of the present application, a nanomaterial is a plurality of particles—in an unbound state, as an aggregate, or as an agglomerate—where, 50% or more of the particles having one or more external dimensions in the size range 1 nm-100 nm; a “non-nano” material does not meet the above definition.

### BACKGROUND OF THE INVENTION

“Sunscreens active ingredients” are defined by the FDA as chemicals that absorbs, reflects, or scatters radiation in the UV range at wavelengths from 290 to 400 nanometers; and can be subdivided into “organic” sunscreens (that absorbUV radiation) and physical (that reflect or scatter UV radiation); the latter include Zinc Oxide and Titanium Dioxide.

Historically, sunscreen formulations containing zinc oxide produced a white appearance caused by back-reflection of visible light from the zinc oxide particles. In response, scattering of light was minimized by reducing the size of the ZnO particles to the nanometer scale, (<100 nm). Additionally, improved transparency of ZnO particles was achieved by minimizing the difference in refractive indices of the particles and the excipient (e.g., inert carrier vehicle) in which the particles are dispersed.

Potential health concerns have been raised about nanoparticles in sunscreens. See, for example, SE Cross et al. “Human Skin Penetration of Sunscreen Nanoparticles: In-vitro Assessment of a Novel Micronized Zinc Oxide Formulation” Skin Pharmacol. Physiol., Vol. 20, pp. 148-154 (2007).

Health concerns have been raised regarding certain sunscreen actives. For example, in June 2012, the California Environmental Protection Agency, Office of Environmental Health Hazard Assessment listed the organic sunscreen benzophenone as a chemical known to the State of California to cause cancer. See also report from the Environmental Working Group published at http://www.ewg.org/sunscreen/report/the-trouble-with-sunscreen-chemicals/, citing two publications (among others) by Janjua N R, et al.: Janjua N R, et al. “Systemic absorption of the sunscreens benzophenone-3, octyl-methoxycinnamate, and 3-(4-methyl-benzylidene) camphor after whole-body topical application and reproductive hormone levels in humans.” J Invest Dermatol. Vol. 123, pp. 57-61 (2004); Janjua N R, et al” Sunscreens in human plasma and urine after repeated whole-body topical application. J Eur Acad Dermatol Venereal. Vol. 22, No. 4, pp. 456-461 (2008).

Health concerns have also been raised with respect to another organic sunscreen-octocrylene. In 2010, in an article entitled “Octocrylene, an emerging photoallergen” published in Archives of Dermatology [Vol. 146, No. 7, pp. at pages 753-757 (2010)], Avenel-Audran M et al. reported that octocrylene appears to be strong allergen leading to contact dermatitis in children and mostly photoallergic contact dermatitis in adults with an often-associated history of photoallergy from ketoprofen. See also, de Groot, AC and DW Roberts, “Contact and photocontact allergy to octocrylene: a review.” Contact Dermatitis, Vol. 70, pp. 193-204. (2014).

Responsive to these concerns, transparent ZnO having average particle sizes larger than 100 nm were developed and marketed, including under the tradename ZinClear by Antaria Limited. See, US Pre-Grant Patent Application Publications 2010/0310871 and 2010/0316582, both abandoned.

Another example of ZnO marketed as having a particle size of greater than 100 nanometers is ARGA-SUN ZnO CLR-P. According to various Technical Data Sheets (TDS), ARGA-SUN ZnO CLR-P have a highly porous structure and an average particle size distribution of less than about 800 nanometers (measured using static laser scattering).

Additionally, the pores of ARGA-SUN ZnO CLR-P particles are described as “infiltrated” (e.g., filled) with excipient, and having a refractive index close to the excipient, resulting in a “significant increase in transparency”.

Because the degree of photoprotection provided by a sunscreen composition—commonly expressed as Sun Protection Factor (SPF), see definition below—is dependent, to a considerable extent, on the formulation, predicting how much sunscreen active ingredients is/are needed to achieve a specific SPF is extremely difficult to predict. http://www.crodapersonalcare.com/home.aspx?s=157&r=265&p=2046. For example, Croda explains “For zinc oxide, 1% solids should give about 1 SPF unit. [Z]inc oxide dispersions contain 60% solids, so a concentration of 10% dispersion in the formulation should give an SPF of about 6.” See, A. Nayyir-Mazhir & K. McNeil, “Formulating broad spectrum UV protection products” Personal Care, pp. 21-24 (November 2013); see also, https://www.makingcosmetics.com/How-Can-1-Get-a-SPF_ep_60.html (accessed on DATE). Technical data sheets from some suppliers of specialty chemicals teach that 1% of ZnO in a formulation can contribute up to about 1.5 units of SPF. See, Technical Data Sheet from Everline for Zana® 10, Zana® 20 & Zana® M (Nov. 25, 2016).

SPF is just one measure of protection for ultraviolet radiation. Photoprotection is also dependent on photostability—the amount (i.e., concentration) by which a sis degraded by one or more photochemical reactions, including photofragmentation, isomerization, tautomerization, photoaddition and substitution reactions, and/or cycloadditions. These photodegradation reactions also generate free radicals, which are associated with adverse health consequences.

Sunscreens are considered “photostable” if the area under a spectral absorption curve after irradiation with a solar simulator is 80% of the area under the spectral absorption curve prior to irradiation.

Increasingly, consumers are demanding photostable sunscreen products that provide broad spectrum protection and have a high SPF, of at least 30, preferably at least 50, and are water resistant, preferably for 80 minutes. Achieving this level of sun protection without using organic sunscreen filters in an aesthetically elegant formulation—one that is non-whitening or oily when applied to skin—are needs that have, and continue, to exist; and are met by compositions of the present invention.

### DETAILED DISCUSSION OF THE INVENTION

The present invention is directed to water-in-oil sunscreen emulsions (i) having an SPF of at least 30, preferably at least 50, that are (ii) “broad spectrum” (according to regulations promulgated by both the US Food and Drug Administration (FDA) and the European Commission; see discussion below), and (iii) photostable and (iv) do not contain an organic sunscreen active that absorbs ultraviolet radiation, but, instead (iv) contain only a single physical sunscreen active ingredient—“non-nano” Zinc Oxide (as defined below).

SPF, the amount of solar energy (UV radiation) required to produce sunburn on protected skin (i.e., with sunscreen applied), is defined in terms of Minimal Erythema Dose (“MED”) according to the formula:

MED (protected skin)/MED (unprotected skin)

More particularly, SPF is the quantity of erythema-effective energy (expressed as Joules per square meter) required to produce the first perceptible, redness reaction with clearly defined borders. MED (protected skin) is the minimal erythemal dose for protected skin after application of 2 milligrams per square centimeter of a sunscreen product. MED (unprotected skin) is the minimal erythema dose for skin to which no sunscreen product has been applied.

“Critical Wavelength” is the wavelength for which the section under the integrated optical density curve starting at 290 nm is equal to 90 percent of the integrated section between 290 nm to 400 nm.

The final FDA Sunscreen Monograph—“Labeling and Effectiveness Testing; Sunscreen Drug Products for Over-the-Counter Human Use,” Vol. 76 Federal Register pp. 35620 (Jun. 17, 2011)—defines a “Broad Spectrum” Sunscreen as having Critical Wavelength of at least 370 nm and a SPF value of 15 or higher.

The test method for determining whether a sunscreen is “water resistant” for 80 minutes is codified by the US Food and Drug Administration at Section 201.327(i)(7) of Volume 21 of the Code of Federal Regulations (“CFR”).

European Commission Recommendation of 22 Sep. 2006 (2006/647/EC) defines a “Broad Spectrum” Sunscreen as having (i) an SPF of more than 6, as determined in accordance with the International Sun Protection Factor Test Method of 2006 (or an equivalent in vitro method), (ii) UVA protection of more than ⅓ of the SPF as determined using the persistent pigment darkening method modified by the French health agency Agence franc; aise de securite sanitaire des produits de sante (or an equivalent in vitro method), and (iii) a critical wavelength of at least 370 nanometers.

### Zinc Oxide

Sunscreen compositions of the present invention contain a single sunscreen active ingredient—“Non-Nano Zinc Oxide”—uncoated particles of Zinc Oxide having an average particle size of greater than about 0.1 microns, preferably having an average particle size of greater than about 0.2 microns, still more preferably having an average particle size of greater than about 0.4 microns, and even more preferably having an average particle size of greater than about 0.5 microns and less than about 0.8 microns.

In preferred embodiments, the Non-Nano Zinc Oxide particles have a porous structure, such that when the pores are filled with an excipient (carrier fluid), the refractive index of the particles is reduced to a value close to that of the excipient. Non-Nano Zinc Oxide particles are commercially available from Argan Co. under the tradename ARGA-SUN ZnO CLR-P.

### SPF-Boosting Emollients

Emollients are cosmetic ingredients that help to maintain the soft, smooth appearance of skin and reduce flaking.

Compositions of the present invention include at least one, preferably at least two, and even more preferably at least three emollients that boost (i.e., increase) SPF, preferably selected from the group consisting of Octyldodecyl Neopentanoate, Butyl Octyl Salycilate, and Cetyl Dimethicone.

Butyloctyl Salicylate, commercially available as HallBrite® BHB from the Hallstar Company is a synthetically produced ester of Salicylic Acid and a branched C_{1 2} alcohol, 2-butyloctanol. Butyloctyl Salycilate has an empirical formula of C₁₉ H₃₀ O₃ and the following structural formula:

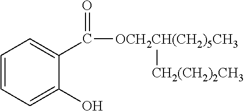

Use of Butyloctyl Salicylate in sunscreen products is described in the following U.S. Pat. Nos. 5,783,173; 5,788,954; 5,849,273; and 6,350,894.

Cetyl Dimethicone is a dimethyl siloxane polymer that conforms to the formula:

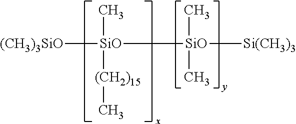

It is commercially available from a number of specialty chemical companies, including under the tradename ABIL® Wax 9801 is commercially available from Evonik Goldschmidt GmbH (Essen, Germany). Technical literature from Evonik recommends ABIL® Wax 9801 for emulsions based “on only physical sunscreens” and claims that the spreading effect (on skin) of ABIL® Wax 9801 will increase the effectivity of UV-filters in emulsions—namely, reducing the level of UV-filters but maintaining SPF.

Compositions of the present invention may, and preferably do, include additional emollients, including, but not limited to Dimethicone and Caprylic/Capric Triglyceride.

Dimethicone is a mixture of fully methylated linear siloxane polymers end blocked with trimethylsiloxy units, that conforms to the formula:

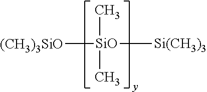

Caprylic/Capric Triglyceride is the mixed triester of glycerin and caprylic and capric acids.

### Photodamage Reducing Ingredients (Ultraviolet and Infrared Radiation)

Photodamage can result from unprotected skin exposure to ultraviolet radiation (UVR) and infrared radiation (IR). Compositions of the present invention include (i) at least one particle (also referred to below as a powder) that increases protection of the skin from UVR-induced photodamage (a “UVR-Protective Powder”) and (ii) at least one particle that blocks, and therefore, provides protection from IR-induced photodamage (an “IR-Protective Powder”). In one aspect of the present invention, by the phrase “increases protection of the skin from UVR-induced photodamage” means increases SPF.

One preferred UVR-Protective Powder is Styrene/Acrylates Copolymer, sold under the tradename Sunspheres from Dow. This polymer of styrene and a monomer consisting of acrylic acid, methacrylic acid or one of their simple esters is manufactured via emulsion polymerization creating what technical literature from Dow describes as “hollow spheres . . . that raise the UV protection over the whole UVA/UVB spectrum and work equally well with organic and inorganic sunscreen actives . . . and is also compatible with a broad range of cosmetic ingredients, including emollients and skin conditioners.”

The technical brochure from Dow continues to state generally, “The ability of SUNSPHERES to boost the efficacy of UVA/UVB filters of formulations allows the formulator to use significantly less active to deliver the same level of SPF.”

Another preferred UVR-Protective Ingredient is Polyhydroxystearic Acid—a polymer of Hydroxystearic Acid (q.v.) that is commercially available under the tradename Dispersun DSP OL 300 from lnnospec Performance Chemicals (Salisbury, N.C.).

According to technical literature from the specialty chemical company lnnospec, Polyhydroxystearic Acid increases UV absorption of sunscreens containing pigments, including by allowing higher concentrations of pigment to be used. Additionally, inclusion of Polyhydroxystearic Acid is described by lnnospec as improving optical transparency and reduce whitening (when finished formulation is applied to skin).

One preferred IR-Protective Particle is ARG-SPHERE NIR-1/1SBA000—a combination of PMMA, Cerium Oxide, and Aluminum Oxide from Argan Co.

Poly(methyl methacrylate), also known in the art as PMMA, is a spherical ultra-fine texturizing powder, available in various sizes, used in emulsions to increase smoothness, fluidity and lubricity. More particularly, PMMA is a polymer of methyl methacrylate, has an empirical formula C₅H₈O₂)ₓ and conforms to the following structure:

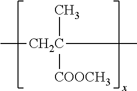

Cerium Oxide (CeO) and Aluminum Oxide (AbO₃) are inorganic oxide compounds.

### Film-Forming Polymer

Compositions of the present invention contain at least one film-forming polymer that imparts water repellency and wash-off resistance.

A preferred film-forming polymer is Acrylates/Dimethicone Copolymer-a copolymer of dimethicone and one or more monomers of acrylic acid, methacrylic acid or one of their simple esters—available from numerous suppliers, including Access Ingredients (South Pasadena, Calif.) under the tradename AccessSlL FF-16.

### Water-in-Oil Emulsifier

Compositions of the present invention are water-in-oil (“W/O”) emulsions. By the term W/O emulsion is to be understood a two-phase mixture of immiscible liquids—an inner phase containing water-soluble (hydrophilic) ingredients dispersed in a continuous outer phase on hydrophobic ingredients (oils, esters, and silicones).

A preferred W/O emulsifier is Polyglyceryl-3 Sorbityl Linseedate is a water-in-silicone emulsifier sold by Sinerga/Argan Co. under the tradename EWOCREAM. More Polyglyceryl-3 Sorbityl Linseedate is the product obtained by the reaction of Linseed Acid (q.v.) with the sorbitol ether of Polyglycerin-3 (q.v.).

### Aesthetic Enhancing Powders

Compositions of the present invention preferably include one powder that enhances skin feel, more preferably more than one powder. In preferred embodiments, the powder reduces the feeling of oiliness that is commonly associated with sunscreen formulations. Ingredients that provide skin-feel improving benefits are referred to in the present application as “Aesthetic Enhancing Powders”.

One Aesthetic Enhancing Powder that is preferably included in compositions of the present invention is “oil-absorbing”, by which is meant the powder has an internal porosity sufficient to absorb oils and esters and, thereby, contribute to a non-greasy skin-feel.

In one preferred embodiment, the oil-absorbing powder has an absorption capacity of at least about 20 ml/100 gas measured by the test method in US Pre-Grant Patent Application 2011/0293543 ¶ [0036]. In another preferred embodiment, oil absorption capacity is determined by weighing approximately 2 to 2.2 g of the powder (sieved through 200 mesh screen in advance) and adding liquid paraffin until loose and dry powder disappears, and expressed according to the following formula: final volume of liquid paraffin absorbed (in cm³)/weight of sample (in grams).

One particularly preferred oil absorbing Aesthetic Enhancing Powder is silica, commercially available from a number of specialty chemical suppliers, including under the tradename Silisphere LS-8H from Argan Co.

In addition, the oil-absorbing powder contributes to homogeneity (i.e., even dispersion of pigments), and also helps to provide soft focus and skin mattifying effects.

The oil-absorbing powder also provides additional processing/manufacturing benefits, including reducing agglomeration (“clumping”).

### Bulking and Binding Agents

Bulking agents are chemically inert, solid ingredients employed as diluents for other solids. Bulking agents can also serve as “extenders” of pigments.

Compositions of the present invention may, and preferably do include a bulking agent, preferably a Hectorite (or a derivative of, or reaction product with, Hectorite).

Disteardimonium Hectorite is the reaction product Hectorite, a montmorillonite mineral that is one the principal constituents of bentonite clay, and Distearyldimonium Chloride, a quaternary ammonium salt that conforms generally to the formula:

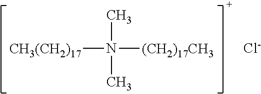

Disteardimonium Hectorite is a rheological additive for low to intermediate polarity organic systems, that increases viscosity, provides thixotropy while preventing syneresis, and prevents pigment settling during storage.

Phenyl Trimethicone is the siloxane polymer that conforms to the formula:

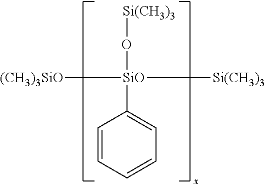

where n is predominantly 1 to 3.

Triethyl Citrate is the triester of ethyl alcohol and citric acid that conforms to the formula:

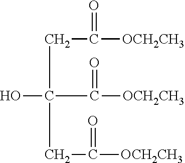 [SMILES: CCOC(=O)CC(O)(CC(=O)OCC)C(=O)OCC]

The mixture with Phenyl Trimethicone (and) Disteardimonium Hectorite (and) Triethyl Citrate under the tradename Bentone Gel PTM V from Elementis.

Literature from Elementis describes Bentone Gel PTM Vas contributing to emulsion stabilization, by providing “thermostable viscosity control” of the emulsion's oil phase, and “excellent suspension of pigments and actives”. In terms of aesthetics, the same document describes Bentone Gel PTM Vas enhancing skin-feel by “masking greasy or tacky components” and imparting a “pleasant residual silkiness to the skin”.

### Environmental Protectants

Compositions of the present invention include one or more Environmental Protectants selected from antioxidants/free-radical quenchers and anti-irritants/anti-inflammatory agents. Three particularly preferred Environmental Protectants are Benzylidene Dimethoxydimethylindanone (C₂₀H₂₀O₃) is an organic compound that conforms to the formula:

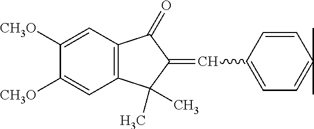

and is commercially available under the tradename SymUrban® from Symrise.

SymUrban® is marketed as an “anti-pollution agent” that reduces environmental damage—namely, collagen degradation, overproduction of melanin and inflammation resulting from exposure to airborne particulate matter found in vehicle exhaust and industrial emissions.

Tocopheryl Acetate is the ester of Tocopherol (q.v.) and acetic acid, and is an article of commerce supplied to the personal care industry by a several companies.

A mixture of Bisabolol and Zingiber officinale (Ginger) Root Extract is commercially available under the tradename SymRelief 100 from Symrise.

Bisabolol is a terpene that conforms to the formula:

### Preservative System

Compositions of the present invention do not use parabens or formaldehyde releasing ingredients.

In preferred embodiments, compositions of the present invention include Hydroxyacetophenone, 1,2 Hexanediol and Caprylyl Glycol.

Hydroxyacetophenone (C₈ H₈O₂) available under the tradename Symsave H from Symrise is the organic compound that conforms to the formula:

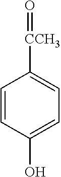

A mixture of 1,2 Hexanediol and Caprylyl Glycol is commercially available under the tradename SymDiol 68 from Symrise.

### Skin Coloring Agent

Compositions of the present invention may, and in certain embodiments are tinted. Such tinted compositions may contain one, preferably, a mixture of iron oxides and micas, or a natural colorant (e.g., caramel).

### FORMULATION EXAMPLES

| INCI Name | Range % |
|---|---|
| Water | 29.00-40.00 |
| Glycerin | 0.10-4.00 |
| Hydroxyacetophenone | 0.10-1.00 |
| Sodium Chloride | 0.10-1.00 |
| Caramel | 0.10-1.00 |
| Caprylic/Capric Triglyceride | 1.00-5.00 |
| Butyl Octyl Salycilate | 10.00-20.00 |
| Polyhydroxystearic Acid | 0.50-2.00 |
| Cetyl Dimethicone | 1.00-5.00 |
| Dimethicone 50 est | 1.00-5.00 |
| Mica and Iron Oxides | 0.10-0.50 |
| Mica and Iron Oxides | 0.50-1.00 |
| Mica and Iron Oxides | 0.0 |
| Phenyl Trimethicone and Disteardimonium Hectorite and | 1.0 |
| Triethyl Citrate | |
| Bisabolol and Zingiber Officinale (Ginger) Root Extr. | 0.01-0.50 |
| Dimethicone (and) Acrylates/Dimethicone Copolymer | 0.10-2.00 |
| Tocopheryl Acetate | 0.10-0.50 |
| 1,2 Hexanediol and Caprylyl Glycol | 0.10-0.50 |
| Benzylidene Dimethoxydimethylindanone | 0.01 |
| Zinc Oxide | 20.00-25.00 |
| Styrene/Acrylates Copolymer | 2.00-6.00 |
| Polyglyceryl-3 Sorbityl Linseedate | 2.00-6.00 |
| Fragrance | 0.01-0.60 |
| Silica | 0.50-5.00 |
| PMMA, Cerium Oxide, Aluminum Oxide | 0.10-2.50 |

1. A broad spectrum, emulsion sunscreen composition having an SPF value of at least 30, wherein the composition
(xx) does not contain an organic sunscreen active that absorbs ultraviolet radiation and
(yy) contains only a physical sunscreen active ingredient, Zinc Oxide, having
(i) a porous structure, such that when pores within Zinc Oxide particles are filled with an excipient, the refractive index of the Zinc Oxide particles is reduced,
(ii) an average particle size of greater than about 100 nanometers, such that the Zinc Oxide is visibly transparent when the sunscreen composition is applied to skin, and
(iii) is water resistant for 80 minutes as determined in accordance with the test method codified at 21 CFR § 201.327(i)(7),
the sunscreen composition being comprised of:
(a) at least one SPF-boosting emollient selected from the group consisting of Octyldodecyl Neopentanoate, Butyl Octyl Salycilate, and Cetyl Dimethicone
(b) at least one powder that provides protection to the skin from photodamage induced by exposure to ultraviolet radiation
(c) at least one powder that provides protection of the skin from photodamage induced by exposure to infrared radiation,
(d) at least one film-forming polymer, and
(e) a water-in-oil emulsifier.

2. The sunscreen composition of claim 1, wherein the at least one powder that provides protection to the skin from photodamage induced by exposure to ultraviolet radiation is selected from the group of Styrene/Acrylates Copolymer or Polyhydroxystearic Acid.

3. The sunscreen composition of claim 1, wherein the at least one powder that provides protection of the skin from photodamage induced by exposure to infrared radiation is a mixture that contains Cerium Oxide and Aluminum Oxide.

4. The sunscreen composition of claim 1, wherein the film-forming polymer is selected from the group consisting of (a) Acrylates/Dimethicone Copolymer and (b) Trimethylsiloxysilicate.

5. The sunscreen composition of claim 1, wherein the water-in-oil emulsifier is Polyglyceryl-3 Sorbityl Linseedate.

6. The sunscreen composition of claim 1, further comprising at least one aesthetic enhancing powder that improves skin feel, is oil-absorbing, imparts silky texture, enhances slip, increases blendability, or reduces the appearance of fine lines and wrinkles on the skin.

7. The sunscreen composition of claim 1, wherein the aesthetic enhancing powder is oil-absorbing.

8. The sunscreen composition of claim 7, wherein the oil-absorbing aesthetic enhancing powder is silica.

9. The sunscreen composition of claim 1, further comprising at least one bulking agent.

10. The sunscreen composition of claim 1, wherein the at least one bulking agent is a Hectorite (or a derivative of, or reaction product with, Hectorite).

11. The sunscreen composition of claim 1, further comprising at least one environmental protectant.

12. The sunscreen composition of claim 11, wherein the at least one environmental protectant is selected from the group of Benzylidene Dimethoxydimethylindanone, Tocopheryl Acetate, Bisabolol and Zingiber officinale Root Extract.

13. The sunscreen composition according to claim 1 having an SPF of at least 50.

14. The sunscreen composition according to claim 1 that is tinted and further comprises one or more iron oxide(s), mica(s). or natural colorants.
